(19) United States
(12) Patent Application Publication
(10) Pub. No.: US 2003/0135211 A1
(43) Pub. Date: Jul. 17, 2003

(54) Intramedullary nail, device for inserting a screw into the same and method thereof

(76) Inventor: Woo Shin Cho, Seoul (KR)

Correspondence Address: Anderson, Kill & Olick, P.C., 1251 Avenue of the Americas, New York, NY, 10020, US

(21) Appl. No.: 10/339,942

(22) Filed: Jan. 10, 2003

(30) Foreign Application Priority Data
Jan. 17, 2002 (KR) 2002-2774

(51) Int. Cl. A61B 17/58

(52) U.S. Cl. 606/62; 606/64; 606/98

(57) ABSTRACT

An intramedullary nail, a device for inserting a screw into the same, and a insertion method thereof. Guide holes are formed between the nail inserted to the medullary cavity and a distal screw guide member. A guide wire having a much smaller diameter than each guide hole is inserted into the guide hole and drilling is then performed along the guide wire. Then, a metal screw is remained into the intramedullary nail to prevent the nail from moving up and down and twisting, thereby allowing easy and accurate insertion of the distal metal screw without an aid of an X-ray imaging device. Therefore, an operation time can be reduced, unnecessary bone damage or infection of a patient can be prevented and a patient and/or surgeon's exposure to X-ray radiations can be considerably reduced.

### BACKGROUND OF THE INVENTION

1. Field of the Invention

The present invention generally relates to an intramedullary nail used for treatment of fractures of long bones such as the femur, tibia or humerus. More particularly, the invention relates to an intramedullary nail into which an interlocking screw can be easily and accurately inserted without using an X-ray imaging device such as a C-arm apparatus. Also, the invention relates to a device for inserting a screw into the intramedullary nail and a method thereof.

2. Description of the Related Art

Recently, intramedullary nails have been generally used for treatment of long bone shaft fractures, since a fracture reduction and an internal fixation could be achieved without exposure of a fracture site, thereby allowing orthopedic treatment to be easily performed. Also, less bleeding and less vascular damage to the fracture site can be expected, achieving better bone union. Since the inserted nail is in the center of a compressive force, a patient can do early mobilization even before proper union of the fractured bone.

Also, there has recently been developed an interlocking intramedullary nail into which a screw can be inserted. In view of many advantages, including stability of a fracture site and less complication, nowadays, most intramedullary nails are being replaced with interlocking intramedullary nails. Through the conventional insertion technique, screws can be easily inserted into the proximal end of the nail. However, for a distal targeting purpose, drilling should be performed using an X-ray imaging device such as a C-arm apparatus, and takes a long learning curve.

As schematically shown in FIG. 3, there is provided a conventional nail with each two screws insertion holes at its proximal end and distal end respectively. The nail may require a screw insertion device with a screw guide member, the device nail may require a screw insertion device with a screw guide member, the device having a hole with the same shape, insertion position and direction as those of the hole of the nail to be inserted and configured to insert metal screws through the hole.

FIG. 3 is a perspective view of a conventional screw insertion device 1 for a nail 10, which will now be described briefly. The screw insertion device 1 includes a screw guide member 20 having a proximal bore 31 and a distal bore 22 to have the same distance and direction as those of proximal and distal bores 11 and 12 of the nail 10 inserted into the medullary cavity, a handle 30 substantially “U” shaped while maintaining a distance between the nail 10 and the screw guide member 20, and a connection member 90 positioned opposite to the handle 30 and having a locking portion 91 configured to align the distal bore 12 of the nail 10 and the distal bore 22 of the screw guide member 20.

In the screw insertion device 1 for the nail 10, an incision of the patient's skin is made. The nail 10 is fixed with a handle 30 and inserted into the medullary cavity of the patient's long bone. Next, a metal screw 40 is inserted into the proximal bore 11 of the nail 10 through the proximal bore 31 of the handle 30 to establish a connection between the long bone and the intramedullary nail 10. Then, while viewing through an X-ray imaging device such as a C-arm apparatus, an operator or surgeon adjusts the locking portion 91 of the connection member 90 up and down, thereby aligning the distal bore 12 of the nail 10 and the distal bore 22 of the screw guide member 20. Thereafter, the metal screw 40 is inserted so as to penetrate the bone and the distal bore 12 to establish a connection between the long bone and the intramedullary nail 10. Finally, the handle 30 is detached from the nail 10.

The above-described interlocking intramedullary nail still requires an X-ray imaging device such as C-arm apparatus when metal screws are inserted into a distal bore. In spite of using such an imaging device, since the hole into which the metal screw is inserted is small, even a small deviation in insertion position or may move up and down or distort when it is intended to be inserted into the distal bore. Thus, the screw guide member and the nail do not coincide with each other in direction and the hole of the nail is too small, making insertion of a metal screw more difficult.

Also, such an inaccurate and difficult insertion procedure may cause damage to the patient's bone to weaken the bone and take a prolonged operation time. Further, patients and surgeons may be exposed to considerable X-ray radiation during the operation.

Therefore, the present invention is devised to solve the above-described problems. A plurality of holes are formed between a proximal bore and a distal bore in the intramedullary nail and a screw guide member. A guide sleeve having a much smaller diameter than each of the holes is inserted into the hole and drilling is performed using the guide sleeve. Then, a metal screw is inserted into the intramedullary nail, thereby easily and accurately inserting the metal screw into the nail while controlling vertical movement and distortion of the nail without an aid of an X-ray imaging device.

### SUMMARY OF THE INVENTION

Therefore, the present invention provides an intramedullary nail which can reduce an operating time and can prevent unnecessary bone damage or infection of a patient, a device for inserting a screw into the same and a screw insertion method thereof.

In accordance with an aspect of the present invention, there is provided a device for inserting one or more screws into a nail upon its insertion into the medullary cavity of a patient comprising a distal screw guide member having proximal and distal disposed at positions and direction corresponding to those of the proximal and distal bores of the nail, and a substantially “U” shaped handle for maintaining a substantially constant distance between the nail and the distal screw guide member. At least one guide hole in said nail and a corresponding guide hole in said distal screw guide member. Also, the device may further include a guide drill sleeve inserted into the guide hole or the distal bore of the distal screw guide member, a guide wire sleeve inserted into the guide drill sleeve, and a guide wire for guiding a drill having a hollow center.

According to another aspect of the present invention, there is provided a method for inserting a screw into an intramedullary nail having a proximal and distal bore in the treatment of a long bone fracture, comprising: fixing the handle to said nail; inserting said nail into the medullary cavity of the long bone of a patient; inserting a metal screw into a proximal bore of the nail through a proximal bore of the handle to establish a connection between the long bone and the intramedullary nail; inserting an assembly of a guide drill sleeve and a guide wire sleeve into a guide hole of the distal screw guide member to make the assembly reach the bone in the vicinity of a guide hole of the nail; inserting a guide wire into the guide hole of the nail through the guide wire sleeve; removing the guide wire sleeve in a state in which the guide wire is inserted into the guide wire sleeve and placing a drill having a hollow center in the guide wire to be inserted into the guide drill sleeve for inserting the drill into the guide hole of the nail; inserting the assembly of the guide drill sleeve and the guide wire sleeve into a distal bore of the distal screw guide member to make the assembly reach the bone in the vicinity of the distal bore of the nail; inserting the guide wire into the guide hole of the nail through the guide wire sleeve; removing the guide wire sleeve in a state in which the guide wire is inserted into the guide wire sleeve and then placing a drill having a hollow center in the guide wire to be inserted into the guide drill sleeve for inserting the drill into the distal bore of the nail; removing the guide wire, the drill and the guide drill sleeve from the distal bores of the nail and distal screw guide member; inserting the metal screw into the distal bore of the nail through the distal bore of the distal screw guide member to establish a connection between the long bone and the intramedullary nail; removing the guide wires in the guide holes, the drill and guide drill sleeve; and detaching the distal screw guide member and the handle from the nail.

### BRIEF DESCRIPTION OF THE DRAWINGS

The above objects and advantages of the present invention will become more apparent by describing preferred embodiments thereof with reference to the attached drawings in which:

FIG. 1 is a perspective view of a nail and a screw insertion device according to the present invention;

FIG. 2 is an exploded perspective view of the nail and screw insertion device shown in FIG. 1; and

FIG. 3 is a perspective view illustrating a conventional nail and a conventional screw insertion device.

### DETAILED DESCRIPTION OF THE INVENTION

The present invention will now be described in more detail with reference to the accompanying drawings, in which like elements are denoted by like reference numerals.

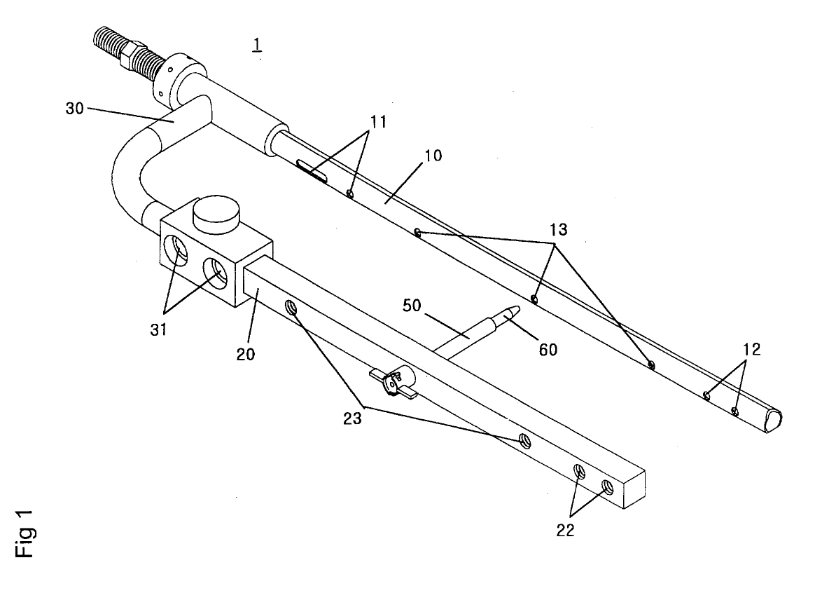

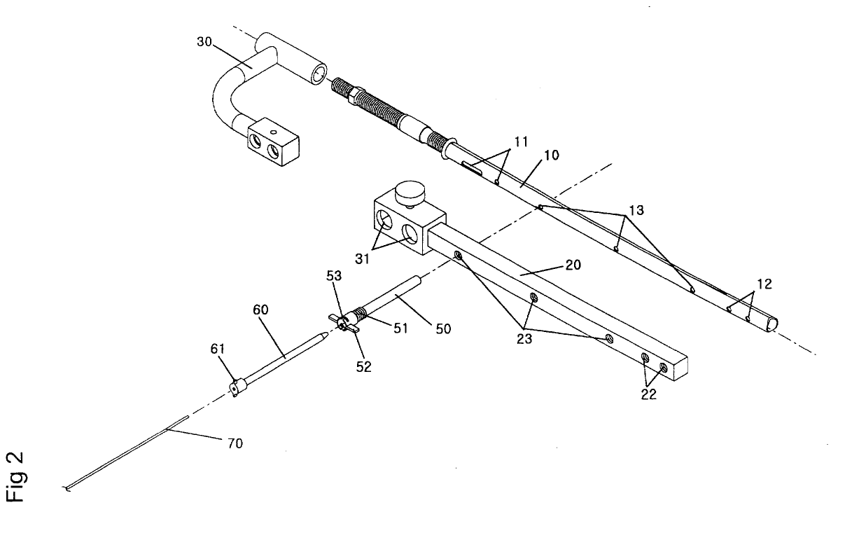

FIG. 1 is a perspective view of a nail and a screw insertion device according to the present invention, and FIG. 2 is an exploded perspective view of the nail and screw insertion device shown in FIG. 1.

As shown in FIGS. 1 and 2, a screw insertion device 1 for a nail according to the present invention includes a cylindrical nail 10 inserted into the medullary cavity of a patient and having a proximal bore 11, a distal bore 12 and at least one guide hole 13; a distal screw guide member 20 having distal bores 22 and at least one guide hole 23 to have the same distance and direction as those of the holes 12 and 13 of the nail 10; and a handle 30 substantially “U” shaped so as to be easily gripped by a surgeon while connecting the nail 10 and the distal screw guide member 20 at a constant distance therebetween. The nail 10, the distal screw guide member 20 and the handle 30 connecting the same to each other are the same as those in the prior art, except that at least one guide hole 13,23 is further formed in 13 of the nail 10; and a handle 30 substantially “U” shaped so as to be easily gripped by a surgeon while connecting the nail 10 and the distal screw guide member 20 at a constant distance therebetween. The nail 10, the distal screw guide member 20 and the handle 30 connecting the same to each other are the same as those in the prior art, except that at least one guide hole 13,23 is further formed in each of the nail 10 and the distal screw guide member 20 so as to have the same distance and direction, and a detailed explanation will not be given. Also, a screw thread is formed inside a guide hole 23 and a distal bore 22 formed in the distal screw guide member 20, thereby achieving a secured connection of a guide drill sleeve 50 to be described later.

The screw insertion device 1 for a nail according to the present invention includes a guide drill sleeve 50 inserted into the guide hole 23 or the distal bore 22 of the distal screw guide member 20, a guide wire sleeve 60 inserted into the guide drill sleeve 50, and a guide wire 70 guiding a drill (not shown) having a hole opened lengthwise.

The guide drill sleeve 50 includes a screw thread 51 at the outer periphery of its one side so as to be threaded with the guide hole 23 and the distal bore 22 of the distal screw guide member 20. Also, a pair of fastening projections 52 are formed at the outer end of the guide drill sleeve 50 to allow the surgeon to easily rotate the guide drill sleeve 50 without a separate tool for inserting the same into the guide hole 23 or the distal bore 22. A pair of locking grooves 53 interacting with locking protrusions 61 of a guide wire sleeve 60 to be described later are formed between the fastening projections 52 for fixedly inserting the guide wire sleeve 60. The drill can pass through the guide drill sleeve 50 lengthwise.

Also, the guide wire sleeve 60 is sized to be placed in the guide drill sleeve 50 and is configured such that the guide wire 70 can pass through the guide wire sleeve 60 longitudinally. A pair of locking protrusions 61 fitted into the locking grooves 53 of the guide drill sleeve 50 so as to fix the guide wire 70 inside guide drill sleeve 50, are formed at one end of the guide wire sleeve 60.

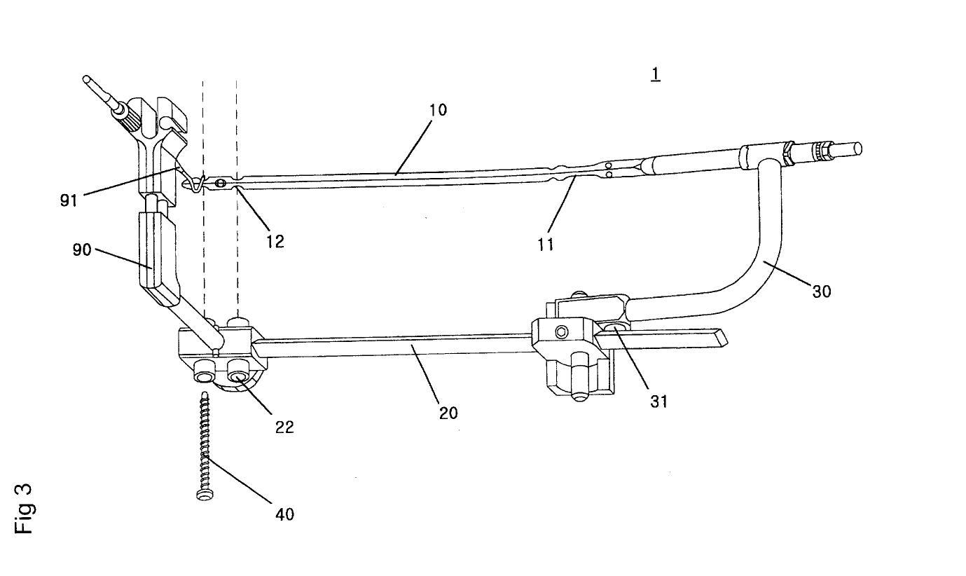

A method of inserting the interlocking metal screw 40 (FIG. 3) into the intramedullary nail 10 using the screw insertion device 1 for a nail according to the present invention will now be described.

First, the patient's skin is incised by a surgeon and the intramedullary nail 10 is fixed to the handle 30. Then, the nail 10 is inserted into the medullary cavity of the long bone. Next, the metal screw 40 is inserted into the proximal bore 11 of the nail 10 through the proximal bore 31 of the handle 30 to establish a connection between the long bone and the intramedullary nail 10 using the metal screw 40 in the conventional manner.

Subsequently, an assembly of the guide drill sleeve 50 and the guide wire sleeve 60 is inserted into at least one guide hole 23 formed at the distal screw guide member 20. After small skin incision, the assembly is inserted until it reaches the bone in the vicinity of the guide hole 13 of the nail 10 corresponding to the guide hole 23. Here, the guide drill sleeve 50 and the guide wire sleeve 60 are assembled by placing the guide wire sleeve 60 inside the guide drill sleeve 50 and locking the locking protrusion 61 of the guide wire sleeve 60 with the locking grooves 53 of the guide drill sleeve 50. Thus, when the surgeon grips the fastening projection 52 of the guide drill sleeve 50 and rotates the same, the guide drill sleeve 50 is screw-fixed to the guide hole 23 and the guide drill sleeve 50 and the guide wire sleeve 60 are both reached to the bone in the vicinity of the guide hole 13 of the nail 10.

Then, the guide wire 70 is inserted into the guide hole 13 of the nail 10 through the guide wire sleeve 60. In a state in which the guide wire 70 is inserted into the guide hole 13, the guide wire sleeve 60 is removed from the guide drill sleeve 50, and the drill having a longitudinal hollow center is then fitted into the guide wire 70. The drill is fitted into the guide drill sleeve 50 connected to the distal screw guide member 20 along the guide wire 70, to be inserted into the guide hole 13 of the nail 10.

Thereafter, the routine from the step of inserting the assembly of the guide drill sleeve 50 and the guide wire sleeve 60 to the step of inserting the drill into the guide hole 13 is sequentially iterated to reach the distal end as many times as the number of guide holes 23, 13 formed in the distal screw guide member 20 and the nail 10. For example, as shown in FIG. 1, each of three guide holes 23, 13 are formed in the distal screw guide member 20 and the nail 10. Thus, the routine is preferably iterated 3 times.

Likewise, the assembly of the guide drill sleeve 50 and the guide wire sleeve 60 is inserted into the distal bore 22 of the distal screw guide member 20 to make the assembly reach the bone in the vicinity of the corresponding distal bore 12 of the nail 10. Subsequently, the guide wire 70 is inserted into the distal bore 12 of the nail 10 through the guide wire sleeve 60. In such a state, the guide wire sleeve 60 is removed from the guide drill sleeve 50 and the drill having a hollow center is fitted into the guide wire 70 for inserting the same into the guide drill sleeve 50. After the drill is inserted into the distal bore 12 of the nail 10, the guide wire 70, the drill and the guide drill sleeve 50 are removed from the distal bores 12 and 22 of the nail 10 and the distal screw guide member 20.

Then, the metal screw 40 is inserted into the distal bore 12 of the nail 10 through the distal bore 22 of the distal screw guide member 20. In such a manner, the long bone and the intramedullary nail 10 are connected to each other by means of the metal screw 40. Finally, the guide wire 70, the drill and the guide drill sleeve 50 are removed from the respective guide holes 13 and 23.

Therefore, the guide wire 70 is inserted through the guide holes 13 and 23 formed in the middle of the nail 10 and the distal screw guide member 20, and drilling is performed along the guide wire 70, thereby easily and accurately inserting the interlocking metal screw 40.

Also, the interlocking metal screw 40 is further inserted into the respective guide holes 13 and 23 from which the drill is removed, thereby achieving reduction and internal fixation of a fracture site in a more stable manner.

As described above, according to the present invention, without an aid of an X-ray imaging device, a drill inserted into a guide hole formed in the middle of a nail and a distal screw guide member prevents the nail from moving up and down and distorting, and promoting easy and accurate insertion of a metal screw. Therefore, the intramedullary nail according to the present invention can reduce an operation time, can prevent unnecessary bone damage or infection and can considerably reduce X-ray radiations to a patient and a surgeon.

In addition, since an interlocking metal screw can be inserted into each guide hole from which a drill is removed, stable internal fixation is possible, thereby reducing a treatment period.

What is claimed is:

1. A nail for insertion into the intramedullary cavity of a patient for treating a fracture comprising a proximal bore, a distal bore and at least one guide hole located between the proximal bore and the distal bore of the nail.

2. A device for inserting one or more screws into an intramedullary nail upon its insertion into the medullary cavity of a patient comprising a screw guide member having proximal and distal bores disposed at positions corresponding to that of the proximal and distal bores of the nail and a substantially “U” shaped handle for maintaining a substantially constant distance between the nail and the screw guide member;
characterized by at least one guide hole in said nail and a corresponding guide hole in said screw guide member;
a guide drill sleeve inserted into the guide hole or the distal bore of the screw guide member;
a guide wire sleeve inserted into the guide drill sleeve; and
a guide wire for guiding a drill having a hollow center.

3. The device according to claim 2, wherein the guide drill sleeve has a screw thread at the outer periphery of its one side, a pair of fastened projections are formed at the outer end of the guide drill sleeve to allow the guide drill sleeve to be easily rotated, and a pair of locking grooves are formed between the fastening projections.

4. The device according to claim 2, wherein the guide wire sleeve is sized to be placed in the guide drill sleeve and has a pair of locking protrusions fitted into the locking groove of the guide drill sleeve.

5. A method for inserting a screw into an intramedullary nail in the treatment of a long bone fracture, said nail having a proximal and distal bore, comprising the steps of:
fixing a handle to said nail with the handle having a proximal bore at a position coincident with the proximal bore in the nail;
inserting said nail into the medullary cavity of the long bone of a patient;
inserting a metal screw into a proximal bore of the nail through a proximal bore of the handle to establish a connection between the long bone and the intramedullary nail
fixing a distal screw guide member with the handle;
inserting an assembly of a guide drill sleeve and a guide wire sleeve into a guide hole of the distal screw guide member to make the assembly reach the bone in the vicinity of a guide hole of the nail;
inserting a guide wire into the guide hole of the nail through the guide wire sleeve;
removing the guide wire sleeve in a state in which the guide wire is inserted into the guide wire sleeve and placing a drill having a hollow center in the guide wire to be inserted into the guide drill sleeve for inserting the drill into the guide hole of the nail;
inserting the assembly of the guide drill sleeve and the guide wire sleeve into a distal bore of the distal screw guide member to make the assembly reach the bone in the vicinity of the distal bore of the nail;
inserting the guide wire into the guide hole of the nail through the guide wire sleeve;
removing the guide wire sleeve in a state in which the guide wire is inserted into the guide wire sleeve and then placing a drill having a hollow center in the guide wire to be inserted into the guide drill sleeve for inserting the drill into the distal bore of the nail;
removing the guide wire, the drill and the guide drill sleeve from the distal bores of the nail and distal screw guide member;
inserting the metal screw into the distal bore of the nail through the distal bore of the distal screw guide member to establish a connection between the long bone and the intramedullary nail;
removing the guide wires in the guide holes, the drill and guide drill sleeve; and
removing the distal screw guide member and the handle.

6. The method according to claim 5, further comprising a step of inserting the metal screw.
